(19) United States
(12) Patent Application Publication
(10) Pub. No.: US 2025/0002429 A1
(43) Pub. Date: Jan. 2, 2025

(54) METHODS AND SYSTEMS FOR REMOVING HOMOGENEOUS CATALYST-RELATED CONTAMINANTS PRESENT IN A HYDROCARBON PRODUCT

(71) Applicant: ExxonMobil Chemical Patents Inc., Baytown, TX (US)

(72) Inventor: Emiel deSmit, Brussels (BE)

(73) Assignee: ExxonMobil Chemical Patents Inc., Baytown, TX (US)

(21) Appl. No.: 18/707,533

(22) PCT Filed: Dec. 2, 2022
(86) PCT No.: PCT/US2022/080797

§ 371 Date: May 3, 2024

Related U.S. Application Data
(60) Provisional application No. 63/288,797, filed on Dec. 13, 2021.

(51) Int. Cl. C07C 7/12; B01J 20/02; B01J 20/28; B01J 20/30; B01J 20/32

(52) U.S. Cl. CPC C07C 7/12; B01J 20/0292; B01J 20/28061; B01J 20/28071; B01J 20/3078; B01J 20/3204; B01J 20/3236

(57) ABSTRACT

Methods and systems for removing homogeneous catalyst-related contaminants present in a hydrocarbon may utilize a supported heteropolyacid scavenger. For example, a process may comprise: contacting a supported heteropolyacid scavenger with a hydrocarbon product comprising a hydrocarbon and 100 ppm or greater of homogeneous catalyst-related contaminants; and adsorbing at least a portion of the homogeneous catalyst-related contaminants onto the supported heteropolyacid scavenger to yield a cleaned hydrocarbon product comprising the hydrocarbon and 10 ppm or less of the homogeneous catalyst-related contaminants.

### CROSS REFERENCE TO RELATED APPLICATIONS

This application is a national phase entry of PCT/US2022/080797 filed Dec. 2, 2022, which claims the benefit of and priority to U.S. Provisional Application No. 63/288,797 filed Dec. 13, 2021, the disclosures of which are incorporated herein by reference.

### FIELD

The present disclosure relates to methods and systems for removing homogeneous catalyst-related contaminants present in a hydrocarbon product.

### BACKGROUND

In many homogeneously catalyzed processes, catalyst quenching and recovery is necessary because the catalyst is still reactive and/or the catalyst concentration is too high to be left in the product for process, product quality, or other reasons. For example, in a metathesis process, residual catalyst species may have an undesirable impact on, for example, product molecule isomerization during product recovery or other downstream process steps. In another example, the color of the hydrocarbon product may be impacted, and often times is significantly impacted, even in the presence of ppm levels of homogeneous catalyst-related contaminants like organometallic catalysts and decomposition products thereof.

Further, many catalyst in homogeneously catalyzed processes comprise a precious metal with a significant price per weight. Therefore, reclaiming and recycling the metal after the catalyst is used may be desirable.

### SUMMARY OF INVENTION

The present disclosure relates to methods and systems for removing homogeneous catalyst-related contaminants present in a hydrocarbon.

A nonlimiting example embodiment is a process comprising: contacting a supported heteropolyacid scavenger with a hydrocarbon product comprising a hydrocarbon and 100 ppm or greater of homogeneous catalyst-related contaminants; and adsorbing at least a portion of the homogencous catalyst-related contaminants onto the supported heteropolyacid scavenger to yield a cleaned hydrocarbon product comprising the hydrocarbon and 10 ppm or less of the homogencous catalyst-related contaminants.

A nonlimiting example embodiment is a system comprising: a hydrocarbon synthesis reactor with a feed line and an effluent line; and the effluent line fluidly coupling the hydrocarbon synthesis reactor to a cleaning unit containing a supported heteropolyacid scavengers. The cleaning unit may be configured for contacting the hydrocarbon product from the hydrocarbon synthesis reactor with the supported heteropolyacid scavengers.

These and other features and attributes of the disclosed methods and systems of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings. The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates a flow diagram of a nonlimiting example method of the present disclosure for removing homogeneous catalyst-related contaminants present in a hydrocarbon product.

FIG. 2 illustrates a diagram of a nonlimiting example system of the present disclosure for removing homogeneous catalyst-related contaminants present in a hydrocarbon product.

### DETAILED DESCRIPTION

The present disclosure relates to methods and systems for removing homogeneous catalyst-related contaminants present in a hydrocarbon product. More specifically, the present disclosure uses heteropolyacid scavengers deposited on a support (also referred to herein as supported heteropolyacid scavengers) to remove homogeneous catalyst-related contaminants present in a hydrocarbon product of a reaction like a metathesis reaction. Typically, said homogeneous catalyst-related contaminants may comprise active organometallic catalyst and/or decomposition products of the organometallic catalyst.

Of particular interest are hydrocarbon products (especially waxy hydrocarbon products comprising one or more C₁₀₊ hydrocarbons) produced using metal carbene catalysts (also referred to as metal carbene complexes or Grubbs catalysts). Said waxy hydrocarbon products are highly viscous, which mitigates the ability to remove the homogeneous catalyst-related contaminants from said products. Current methods use water washing, which often uses more than 20 equivalents of water to waxy hydrocarbon product, to remove the homogeneous catalyst-related contaminants. Further, the water introduces additional issues in downstream processing.

Other solution-based methods for cleaning waxy hydrocarbon products may use polar molecules, bases, or molecular scavengers, but similar to the water, the liquid is difficult to completely remove and causes issues with downstream processing of the waxy hydrocarbon products.

Solid materials like silica gel, activated carbon, and mesoporous silicates have also been used, but the uptake of homogeneous catalyst-related contaminants is not efficient and the amount of solid material needed can be extensive.

Advantageously, the heteropolyacid scavengers of the supported heteropolyacid scavengers described herein provides the adsorption efficacy of the solution-based scavengers while the support of the supported heteropolyacid scavengers mitigates the residual liquid issues with the solution-based scavengers.

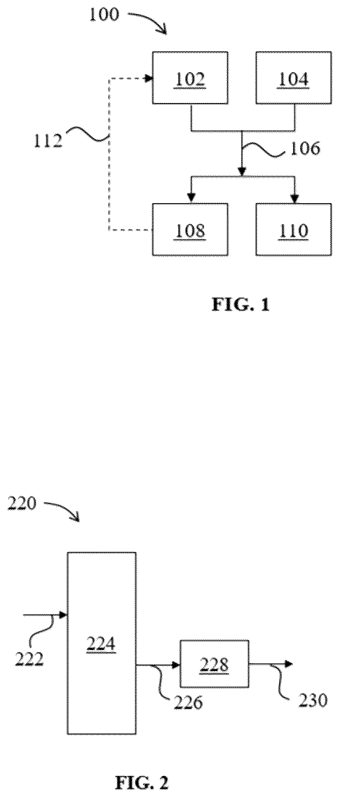

FIG. 1 illustrates flow diagram of a method 100 of the present disclosure. The method includes contacting 106 supported heteropolyacid scavengers 102 and a hydrocarbon product 104. The hydrocarbon product 104 may comprise a hydrocarbon and homogeneous catalyst-related contaminants. The homogeneous catalyst-related contaminants may comprise a metal carbene catalyst and/or a metal carbene catalyst decomposition product. The homogeneous catalyst-related contaminants may comprise one or more metal of: Cu, Ru, Fe, Co, Ni, Pd, Pt, Rh, and W. The homogeneous catalyst-related contaminants may be quantified by metal concentration. The metal concentration (e.g., cumulative concentration of metal (e.g., Cu, Ru, Fc, Co, Ni, Pd, Pt, Rh, and W) that can be attributed to the homogencous catalyst) may be at least 0.1 ppm (or about 0.1 ppm to about 500 ppm or more, or about 0.1 ppm to about 20 ppm, or about 5 ppm to about 100 ppm, or about 10 ppm to about 200 ppm, or about 200 ppm to about 500 ppm) of the hydrocarbon product 104.

Examples of supports of the supported heteropolyacid scavengers may include, but are not limited to, alumina, modified activated alumina, silica, mesoporous silica, titania, metal-organic frameworks, zeolites, activated carbon, modified activated carbon, the like, and any combination thereof. Examples of heteropolyacid scavengers of the supported heteropolyacid scavengers may include, but are not limited to, silicotungstic acid, phosphomolybdic acid, phosphotungstic acid, the like, and any combination thereof. Of particular interest are heteropolyacid structures based on the Keggin ion XM₁₂O₄₀ structure, where X is a main group element such as silicon or phosphorous and M is a metal ion such as W or Mo. These can be partially substituted by other transition metals such as V, Co, Fe, Zn, and others to tune the properties of the heteropolyacid phase. Of specific interest are heteropolyacids containing phosphorous. Furthermore, heteropolyacid phase properties such adsorbent effectiveness, solubility and strength of anchoring to the support material can be improved by the (partial) substitution of the hydrogen counterions by counterions such as lithium, sodium, potassium, cesium, ammonium, and/or cerium. A nonlimiting example supported heteropolyacid scavenger may be an activated carbon-supported phosphotungstic acid.

The hydrocarbon of the hydrocarbon product may have a melting point of about −200° C. to about 80° C. (or about −200° C. to about −50° C., or about −150° C. to about 0° C., or about −40° C. to about 40° C., or about 25° C. to about 60° C., or about 45° C. to about 70° C., or about 50° C. to about 80° C.).

The hydrocarbon of the hydrocarbon product may comprise one or more C₂-C₁₀ hydrocarbons (or one or more C₂-C₆ hydrocarbons, or one or more C₄-C₁₀ hydrocarbons). The hydrocarbon of the hydrocarbon product may comprise one or more C₁₂-C₂₄ hydrocarbons (or one or more C₁₂-C₁₈ hydrocarbons, or one or more C₁₆-C₂₄ hydrocarbons). The hydrocarbon of the hydrocarbon product may comprise one or more C₂₆₊ hydrocarbons. The hydrocarbon of the hydrocarbon product may comprise one or more C₂-C₁₀ hydrocarbons (or one or more C₂-C₆ hydrocarbons, or one or more C₄-C₁₀ hydrocarbons) and one or more C₁₂-C₂₄ hydrocarbons (or one or more C₁₂-C₁₈ hydrocarbons, or one or more C₁₆-C₂₄ hydrocarbons). The hydrocarbon of the hydrocarbon product may comprise one or more C₂-C₁₀ hydrocarbons (or one or more C₂-C₆ hydrocarbons, or one or more C₄-C₁₀ hydrocarbons), one or more C₁₂-C₂₄ hydrocarbons (or one or more C₁₂-C₁₈ hydrocarbons, or one or more C₁₆-C₂₄ hydrocarbons), and one or more C₂₆₊ hydrocarbons. The hydrocarbon of the hydrocarbon product may comprise one or more C₁₂-C₂₄ hydrocarbons (or one or more C₁₂-C₁₈ hydrocarbons, or one or more C₁₆-C₂₄ hydrocarbons) and one or more C₂₆₊ hydrocarbons.

The hydrocarbon of the hydrocarbon product may comprise at least one linear internal olefin, at least one linear terminal olefin, at least one linear terminal and internal olefin, at least one branched internal olefin, at least one branched terminal olefin, at least one branched terminal and internal olefin, at least one (alkyl) aromatic, at least one (iso) parrafin, at least one naphthenic, at least one oxygenated hydrocarbon (e.g., primary alcohols, secondary alcohols, aldehydes, and acids), or any combination thereof.

For example, a linear internal olefin may have a structure according to Formula I:

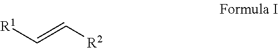

wherein R¹ and R² are independently selected alkyl groups having from 6 to 50 carbon atoms (or 10 to 28 carbon atoms, or 10 to 24 carbon atoms).

The hydrocarbon product may be a product stream from a linear alpha olefin synthesis process.

During contacting 106, at least a portion of the homogencous catalyst-related contaminants may adsorb onto the supported heteropolyacid scavengers 102 to yield (a) a cleaned hydrocarbon product 110 comprising the hydrocarbon and 10 ppm or less of the homogeneous catalyst-related contaminants and (b) supported heteropolyacid scavengers 108 with homogeneous catalyst-related contaminants adsorbed thereto. During the contacting a weight ratio of the homogeneous catalyst-related contaminants to the supported heteropolyacid scavenger may be about 1:5 to about 1:1000 (about 1:5 to about 1:100, or about 1:50 to about 1:250, or about 1:200 to about 1:600, or about 1:500 to about 1:1000).

Contacting 106 occurs while the hydrocarbon is in a fluid state. Contacting 106 may be achieved by mixing the supported heteropolyacid scavengers 102 and the hydrocarbon product 104, flowing the hydrocarbon product 104 over the supported heteropolyacid scavengers 102 (e.g., flowing the hydrocarbon product 104 through a packed bed of the supported heteropolyacid scavengers 102), suspending the adsorbent in a continuous stirred tank reactor followed by filtration, applying the adsorbent in a fluidized or moving adsorbent bed, applying the adsorbent as part of a adsorptive distillation process, or any other suitable contacting process.

The hydrocarbon of the hydrocarbon product may have a melting point of about 25° C. to about 80° C. (or about 25° C. to about 60° C., or about 45° C. to about 70° C., or about 50° C. to about 80° C.). Contacting 106 may be performed at a temperature of about 25° C. to about 550° C. (or about 25° C. to about 100° C., or about 40° C. to about 90° C., or about 75° C. to about 150° C.).

The resultant (a) cleaned hydrocarbon product 110 and (b) supported heteropolyacid scavengers 108 with homogeneous catalyst-related contaminants adsorbed thereto should be separable. In some instances, the contacting 106 process may result in separated cleaned hydrocarbon product 110 and supported heteropolyacid scavengers 108. For example, flowing the hydrocarbon product 104 through a packed bed of the supported heteropolyacid scavengers 102 allows for the supported heteropolyacid scavengers 108 with homogeneous catalyst-related contaminants adsorbed thereto to stay in the packed bed and the cleaned hydrocarbon product 110 to be an effluent stream from the backed bed. However, if the contacting 106 process does not inherently result in separated cleaned hydrocarbon product 110 and supported heteropolyacid scavengers 108, a separation step (not illustrated) may be included in the method. For example, if contacting is performed via mixing, the mixture may be settled and filtered to remove the supported heteropolyacid scavengers 108 with homogeneous catalyst-related contaminants adsorbed thereto from the cleaned hydrocarbon product 110.

Optionally, the method 100 may further include regenerating 112 the supported heteropolyacid scavenger 108 having the homogencous catalyst-related contaminants adsorbed thereto so as to remove at least some of the homogeneous catalyst-related contaminants adsorbed to the supported heteropolyacid scavenger 108. This yields supported heteropolyacid scavengers that may be used as the supported heteropolyacid scavengers 102 in further processing. Regeneration may be achieved by washing with one or more solvents (e.g., water, alcohol, non-polar solvent, and the like) and/or burning away hydrocarbon contaminants on the surface of the supported heteropolyacid scavenger 108.

Optionally, the method 100 may further include, as an alternative to regenerating 112, reclaiming the metal of the homogeneous catalyst-related contaminants adsorbed on the supported heteropolyacid scavenger 108. Reclamation may be achieved by removing the supported heteropolyacid scavenger 108 and other non-metal contaminants by burning any hydrocarbon contaminants and/or dissolving the support with an acid or basc.

FIG. 2 is a flow diagram of a system 220 of the present disclosure. The system 220 includes a hydrocarbon synthesis reactor 224 with one or more feed lines 222 and one or more effluent lines 226. Depending on the hydrocarbon synthesis being performed, the feed lines 222 may be useful for introducing reactants, diluents, catalysts, the like, and any combination thereof into the hydrocarbon synthesis reactor 224, and the effluent lines 226 may useful for transporting hydrocarbon product, recycle streams, the like, and any combination thereof from hydrocarbon synthesis reactor 224. In the illustrated system 220, an effluent line 226 fluidly couples the hydrocarbon synthesis reactor 224 to a cleaning unit 228 that is configured for contacting the hydrocarbon product with the supported heteropolyacid scavengers. For example, the cleaning unit may comprise one or more fixed beds of supported heteropolyacid scavengers through which the hydrocarbon product flows. In another example, the cleaning unit may comprise a tank for mixing the supported heteropolyacid scavengers and the hydrocarbon product flows and a filtration apparatus for separating the supported heteropolyacid scavengers and the hydrocarbon product.

The cleaning unit 228 has an effluent line 230 for conveying the cleaned hydrocarbon product from the cleaning unit 228 to another destination (e.g., a storage tank, a transportation vessel, or other equipment for processing the hydrocarbon product).

The cleaning unit 228 may include two or more parallel lines, apparatuses, or flows for contacting the hydrocarbon product with the supported heteropolyacid scavengers. This may allow for taking one or more of the parallel lines offline for regeneration of the supported heteropolyacid scavengers with minimal, if any, effect to the cleaning of the hydrocarbon product.

The lines illustrated in FIG. 2 provide simple flow or conveyance illustrations and may, in reality, be composed of multiple lines and have equipment (e.g., sensors, pumps, and the like) located along said lines.

### Additional Embodiments

Embodiment 1. A process comprising: contacting a supported heteropolyacid scavenger with a hydrocarbon product comprising a hydrocarbon and 100 ppm or greater of homogeneous catalyst-related contaminants; and adsorbing at least a portion of the homogeneous catalyst-related contaminants onto the supported heteropolyacid scavenger to yield a cleaned hydrocarbon product comprising the hydrocarbon and 10 ppm or less of the homogeneous catalyst-related contaminants.

Embodiment 2. The process of Embodiment 1, wherein the homogeneous catalyst-related contaminants comprise a metal and the metal is present in the hydrocarbon product at about 0.1 ppm to about 500 ppm.

Embodiment 3. The process of any of Embodiments 1-2, wherein the homogeneous catalyst-related contaminants comprises a metal carbene catalyst and/or a metal carbene catalyst decomposition product.

Embodiment 4. The process of any of Embodiments 1-3, further comprising: regenerating the supported heteropolyacid scavenger having the homogeneous catalyst-related contaminants adsorbed thereto.

Embodiment 5. The process of any of Embodiments 1-3, wherein the homogeneous catalyst-related contaminants comprise a metal, and wherein the method further comprises: reclaiming the metal from the supported heteropolyacid scavenger having the homogeneous catalyst-related contaminants adsorbed thereto.

Embodiment 6. The process of any of Embodiments 1-5, wherein the hydrocarbon has a melting point of about −200° C. to about 80° C.

Embodiment 7. The process of any of Embodiments 1-6, wherein the hydrocarbon comprises one or more C₂-C₁₀ hydrocarbons.

Embodiment 8. The process of any of Embodiments 1-7, wherein the hydrocarbon comprises one or more C₁₂-C₂₄ hydrocarbons.

Embodiment 9. The process of any of Embodiments 1-8, wherein the hydrocarbon comprises one or more C₂₆₊ hydrocarbons.

Embodiment 10. The process of any of Embodiments 1-9, wherein the hydrocarbon comprises at least one linear internal olefin.

Embodiment 11. The process of Embodiment 10, wherein the linear internal olefin has a structure represented by Formula I wherein R¹ and R² are independently selected alkyl groups having from 6 to 50 carbon atoms.

Embodiment 12. The process of Embodiment 11, wherein R¹ and R² are independently selected alkyl groups having from about 10 to about 28 carbon atoms.

Embodiment 13. The process of any of Embodiments 1-12, wherein the homogeneous catalyst-related contaminants comprises one or more of: Cu, Ru, Fe, Co, Ni, Pd, Pt, Rh, or W.

Embodiment 14. The process of any of Embodiments 1-13, wherein during the contacting a weight ratio of the homogeneous catalyst-related contaminants to the supported heteropolyacid scavenger ranges from 1:5 to 1:1000.

Embodiment 15. The process of any of Embodiments 1-14, wherein the supported heteropolyacid scavenger is supported on a substrate comprising alumina, modified activated alumina, silica, mesoporous silica, titania, metal-organic framework, zeolite, activated carbon, modified activated carbon, or any combination thereof.

Embodiment 16. The process of any of Embodiments 1-15, wherein the supported heteropolyacid scavenger comprises a heteropolyacid selected from the group consisting of: silicotungstic acid, phosphomolybdic acid, phosphotungstic acid, and any combination thereof.

Embodiment 17. The process of any of Embodiments 1-16, wherein the supported heteropolyacid scavenger comprises a heteropolyacid of the Keggin structure.

Embodiment 18. The process of any of Embodiments 1-16, wherein the supported heteropolyacid scavenger comprises a heteropolyacid of Keggin structure with a fraction of the hydrogen atoms substituted by a group 1 alkali metal ion, an ammonium ion, a cerium ion, or a combination thereof.

Embodiment 19. The process of any of Embodiments 1-14, wherein the supported heteropolyacid scavenger is an activated carbon-supported phosphotungstic acid.

Embodiment 20. The process of any of 1-19, wherein the contacting is performed at a temperature of about 25° C. to about 150° C.

Embodiment 21. A system comprising: a hydrocarbon synthesis reactor with a feed line and an effluent line; and the effluent line fluidly coupling the hydrocarbon synthesis reactor to a cleaning unit containing a supported heteropolyacid scavengers.

Embodiment 22. The system of Embodiment 21, wherein the cleaning unit is configured for contacting the hydrocarbon product from the hydrocarbon synthesis reactor with the supported heteropolyacid scavengers.

Embodiment 23. The system of any of Embodiments 21-22, wherein the supported heteropolyacid scavengers are contained in a fixed bed contained in the cleaning unit.

Embodiment 24. The system of any of Embodiments 21-22, wherein the supported heteropolyacid scavengers are contained in a tank within the cleaning unit, wherein the tank is configured for mixing the supported heteropolyacid scavengers and the hydrocarbon product, and wherein the cleaning unit further comprises a filtration apparatus configured for separating the supported heteropolyacid scavengers and the hydrocarbon product.

Embodiment 25. The system of any of Embodiments 21-24, wherein the cleaning unit comprises an effluent line for conveying the cleaned hydrocarbon product from the cleaning unit to another destination.

Embodiment 26. The system of any of Embodiments 21-25, wherein the cleaning unit comprises two or more parallel lines, parallel apparatuses, or parallel flows for contacting the hydrocarbon product with the supported heteropolyacid scavengers.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

To facilitate a better understanding of the embodiments of the present invention, the following examples of preferred or representative embodiments are given. In no way should the following examples be read to limit, or to define, the scope of the invention.

### EXAMPLES

Adsorbent Preparation. Each of the adsorbents described in Tables 1 and 2 were pretreated for at least 12 hour under a nitrogen flow at the elevated temperature provided in Table 3.

TABLE 1

| Sample | | Name | Description |
|---|---|---|---|
| 1 | (inv.) | MSA-FC C1 (BASF) | ~6 wt. % Phosphotungstic |
| | | (powder) | Acid Scavenger on |
| | | | Activated Carbon |
| 2 | (comp.) | AZ300 (UOP) (extrudate) | Formulated Al₂O₃/Zeolite |
| 3 | (comp.) | F-200 (BASF) (extrudate) | Activated Alumina |
| 4 | (comp.) | D5 (Cabot) (extrudate) | Activated carbon |
| 5 | (comp.) | D5 (Cabot) (powder) | Activated carbon |

TABLE 2

| | | | | t-Plot | | |
|---|---|---|---|---|---|---|
| | | BET | t-Plot | External | t-Plot | Total |
| | | Surface | Micropore | Surface | micropore | Pore |
| | | Area | Area | Area | volume | Volume |
| Sample | Adsorbent | (m²/g) | (m²/g) | (m²/g) | (cm³/g) | (cm³/g) |
| 1 (inv.) | MSA-FC C1 | 841 | 667 | 174 | 0.32 | 0.646 |
| | (BASF) | | | | | |
| | (powder) | | | | | |
| 2 (comp.) | AZ300 (UOP) | 427 | 205 | 221 | 0.09 | 0.375 |
| | (extrudate) | | | | | |
| 3 (comp.) | F-200 (BASF) | 335 | 134 | 201 | 0.09 | 0.391 |
| | (extrudate) | | | | | |
| 4 (comp.) | D5 activated | 1032 | 1021 | 11 | 0.41 | 0.482 |
| | carbon (Cabot) | | | | | |
| | (extrudate) | | | | | |
| 5 (comp.) | D5 activated | 916 | 901 | 14 | 0.36 | 0.433 |
| | carbon (Cabot) | | | | | |
| | (powder) | | | | | |

TABLE 3

| | | | Elevated |
|---|---|---|---|
| Sample | | Adsorbent | Temperature |
| 1 | (inventive) | MSA-FC C1 (BASF) (powder) | 110° C. |
| 2 | (comparative) | AZ300 (UOP) (extrudate) | 400° C. |
| 3 | (comparative) | F-200 (BASF) (extrudate) | 400° C. |
| 4 | (comparative) | D5 activated carbon | 200° C. |
| | | (Cabot) (extrudate) | |
| 5 | (comparative) | D5 activated carbon | 110° C. |
| | | (Cabot) (powder) | |

Batch Adsorption Experiments. About 40 grams of hydrocarbon wax (specifically, C₂₆-C₃₄ metathesis wax) was mixed with about 6 mg of second generation Grubbs catalyst (Formula II below) to produce samples of hydrocarbon product comprising homogeneous catalyst-related contaminants.

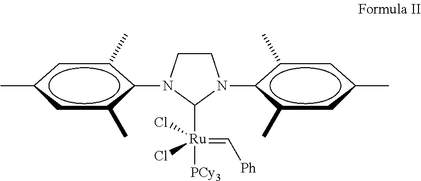

Batch adsorption experiments were performed using a 100 mL Parr 4598 autoclave reactor. In separate experiments, the hydrocarbon product comprising homogeneous catalyst-related contaminants was loaded with about 1 wt % of adsorbent, mixed in the reactor for about 24 hours at 150 rpm and about 65° C. A stirring basket was used for the facile separation of the adsorbent extrudates from the hydrocarbon mixture. For the powder-based samples, after adsorption, the adsorbent was settled from the fluid before sampling the hydrocarbon and adsorbent phases. The resultant hydrocarbon product and adsorbents were analyzed with inductively coupled plasma-optical emission spectroscopy (ICP-OES) with results presented in Tables 4 and 5, respectively.

TABLE 4
Hydrocarbon Product Analysis

| | | | Ru after | |
|---|---|---|---|---|
| | | Ru in starting | adsorption | |
| Sample | | material (wt ppm) | (wt ppm) | Ru adsorbed (wt %) |
| 1 | (inv.) | 24 | 3 | 88 |
| 2 | (comp.) | 18 | 14 | 22 |
| 3 | (comp.) | 21 | 21 | ~0 |
| 4 | (comp.) | 32 | 29 | 9 |
| 5 | (comp.) | 23 | 15 | 35 |

TABLE 5
Adsorbent Analysis

| | | |
|---|---|---|
| | Ru in starting material | Ru after adsorption |
| Sample | (wt ppm) | (wt ppm) |

| | | | |
|---|---|---|---|
| 1 | (inv.) | 6 | 527 |
| 2 | (comp.) | 0 | 194 |
| 3 | (comp.) | 0 | 29 |
| 4 | (comp.) | 0 | 47 |
| 5 | (comp.) | 0 | 298 |

This example illustrates that a supported heteropolyacid scavengers is more effective at removing the homogeneous catalyst-related contaminants, as evidenced by the Ru concentration changes, than an adsorbent without heteropolyacid scavengers thereon.

In a second example, a silica-supported partially cesium substituted phosphotungstic acid adsorbent was prepared by incipient wetness impregnation. The phosphotungstic acid (HPW) phase was synthesized by acid condensation from sodium tungstate and sodium phosphate. To obtain the Cesium-substituted HPW phase, the silica material was first impregnated with cesium carbonate by aqueous incipient wetness onto silica extrudates (PQ Chemicals 1/16″ extrudates, 229 m² g⁻¹ surface area and 0.82 g cm-3 total pore volume) dried at 110° C. and calcined at 300° C. Following this, the phosphotungstic acid (HPW) phase was introduced by impregnation using aqueous incipient wetness impregnation, dried at 110° C. and calcined at 300° C. After preparation, the extrudates were crushed to powder before testing. The description and textural properties for the materials used in Example 2 are summarized in Table 6 and 7, respectively.

TABLE 6

| Sample | | Name | Description |
|---|---|---|---|
| 6 | (inv.) | 40 wt. % | 40 wt. % Cesium-substituted |
| | | Cs_{2.25}H_{0.75}PW₁₂O₄₀ | Phosphotungstic |
| | | on SiO₂ (powder) | Acid on Silica |
| 7 | (comp.) | SiO₂ (PQ Chemicals) | Silica support |
| | | (powder) | |

TABLE 7

| | | | | t-Plot | | |
|---|---|---|---|---|---|---|
| | | BET | t-Plot | External | t-Plot | Total |
| | | Surface | Micropore | Surface | micropore | Pore |
| | | Area | Area | Area | volume | Volume |
| Sample | Adsorbent | (m²/g) | (m²/g) | (m²/g) | (cm³/g) | (cm³/g) |
| 6 (inv.) | 40 wt. % | 109 | 52 | 57 | 0.02 | 0.378 |
| | Cs_{2.25}H_{0.75}PW₁₂O₄₀ | | | | | |
| | on SiO₂ (powder) | | | | | |
| 7 (comp.) | SiO₂ (PQ | 229 | 32 | 197 | 0.01 | 0.821 |
| | Chemicals) | | | | | |
| | (powder) | | | | | |

The materials were tested for their performance in the removal of residual homogeneous catalyst contaminants at the same conditions and using the same equipment as in Example 1. Samples 6 and 7 were pretreated for at least 12 hour under a nitrogen flow at 200° C. The hydrocarbon product comprising homogeneous catalyst-related (Ru) contaminants was loaded with about 1 wt. % of adsorbent in the powdered form, mixed in the reactor for about 24 hours at 150 rpm at 65° C. After adsorption, the adsorbent was settled from the hydrocarbon product before sampling the hydrocarbon and adsorbent phases. The resultant hydrocarbon products were analyzed with inductively coupled plasma-optical emission spectroscopy (ICP-OES). The results from the example are presented in Table 8.

The example shows that, even though part of the accessible surface area and pore volume was lost upon supporting the cesium-substituted heteropolyacid based catalyst on silica, the effective removal of the Ru contaminants form the hydrocarbon phase is improved for the silica supported Cs-substituted HPW sample (Sample 6) compared to the bare silica support (Sample 7).

TABLE 8
Hydrocarbon Product Analysis

| | | | Ru after | |
|---|---|---|---|---|
| | | Ru in starting | adsorption | |
| Sample | | material (wt ppm) | (wt ppm) | Ru adsorbed (wt %) |
| 6 | (inv.) | 22 | 8 | 64 |
| 7 | (comp.) | 21 | 10 | 51 |

Therefore, the present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A process comprising:
contacting a supported heteropolyacid scavenger with a hydrocarbon product comprising a hydrocarbon and 100 ppm or greater of homogeneous catalyst-related contaminants; and
adsorbing at least a portion of the homogeneous catalyst-related contaminants onto the supported heteropolyacid scavenger to yield a cleaned hydrocarbon product comprising the hydrocarbon and 10 ppm or less of the homogeneous catalyst-related contaminants.

2. The process of claim 1, wherein the homogeneous catalyst-related contaminants comprise a metal and the metal is present in the hydrocarbon product at about 0.1 ppm to about 500 ppm.

3. The process of claim 1, wherein the homogeneous catalyst-related contaminants comprises a metal carbene catalyst and/or a metal carbene catalyst decomposition product.

4. The process of claim 1, further comprising:
regenerating the supported heteropolyacid scavenger having the homogeneous catalyst-related contaminants adsorbed thereto.

5. The process of claim 1, wherein the homogeneous catalyst-related contaminants comprise a metal, and wherein the method further comprises: reclaiming the metal from the supported heteropolyacid scavenger having the homogeneous catalyst-related contaminants adsorbed thereto.

6. The process of claim 1, wherein the hydrocarbon has a melting point of about −200° C. to about 80° C.

7. The process of claim 1, wherein the hydrocarbon comprises one or more C₂-C₁₀ hydrocarbons.

8. The process of claim 1, wherein the hydrocarbon comprises one or more C₁₂-C₂₄ hydrocarbons.

9. The process of claim 1, wherein the hydrocarbon comprises one or more C₂₆₊ hydrocarbons.

10. The process of claim 1, wherein the hydrocarbon comprises at least one linear internal olefin.

11. The process of claim 10, wherein the linear internal olefin has a structure represented by 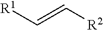
wherein R¹ and R² are independently selected alkyl groups having from 6 to 50 carbon atoms.

12. The process of claim 11, wherein R¹ and R² are independently selected alkyl groups having from about 10 to about 28 carbon atoms.

13. The process of claim 1, wherein the homogeneous catalyst-related contaminants comprises one or more of: Cu, Ru, Fe, Co, Ni, Pd, Pt, Rh, or W.

14. The process of claim 1, wherein during the contacting a weight ratio of the homogeneous catalyst-related contaminants to the supported heteropolyacid scavenger ranges from 1:5 to 1:1000.

15. The process of claim 1, wherein the supported heteropolyacid scavenger is supported on a substrate comprising alumina, modified activated alumina, silica, mesoporous silica, titania, metal-organic framework, zeolite, activated carbon, modified activated carbon, or any combination thereof.

16. The process of claim 1, wherein the supported heteropolyacid scavenger comprises a heteropolyacid selected from the group consisting of: silicotungstic acid, phosphomolybdic acid, phosphotungstic acid, and any combination thereof.

17. The process of claim 1, wherein the supported heteropolyacid scavenger comprises a heteropolyacid of the Keggin structure.

18. The process of claim 1, wherein the supported heteropolyacid scavenger comprises a heteropolyacid of Keggin structure with a fraction of the hydrogen atoms substituted by a group 1 alkali metal ion, an ammonium ion, a cerium ion, or a combination thereof.

19. The process of claim 1, wherein the supported heteropolyacid scavenger is an activated carbon-supported phosphotungstic acid.

20. A system comprising:
a hydrocarbon synthesis reactor with a feed line and an effluent line; and
a cleaning unit containing a supported heteropolyacid scavengers, wherein
the effluent line fluidly couples the hydrocarbon synthesis reactor to the cleaning unit,
the cleaning unit is configured to contact the supported heteropolyacid scavenger with a hydrocarbon product from the effluent line, the hydrocarbon product including a hydrocarbon and 100 ppm or greater of homogeneous catalyst-related contaminants, and
the supported heteropolyacid scavenger adsorbs at least a portion of the homogeneous catalyst-related contaminants to yield a cleaned hydrocarbon product including the hydrocarbon and 10 ppm or less of the homogeneous catalyst-related contaminants.

21. The system of claim 20, wherein the supported heteropolyacid scavenger is supported on a substrate comprising alumina, modified activated alumina, silica, mesoporous silica, titania, metal-organic framework, zeolite, activated carbon, modified activated carbon, or any combination thereof.

22. The system of claim 20, wherein the supported heteropolyacid scavenger comprises a heteropolyacid selected from the group consisting of: silicotungstic acid, phosphomolybdic acid, phosphotungstic acid, and any combination thereof.

23. The system of claim 20, wherein the supported heteropolyacid scavenger comprises a heteropolyacid of the Keggin structure.

24. The system of claim 20, wherein the supported heteropolyacid scavenger comprises a heteropolyacid of Keggin structure with a fraction of the hydrogen atoms substituted by a group 1 alkali metal ion, an ammonium ion, a cerium ion, or a combination thereof.

25. The system of claim 20, wherein the supported heteropolyacid scavenger is an activated carbon-supported phosphotungstic acid.
